(19) United States
(12) Patent Application Publication
(10) Pub. No.: US 2025/0160935 A1
(43) Pub. Date: May 22, 2025

(54) HEAT TRANSFER TECHNIQUES IN HEAT-GENERATING MEDICAL DEVICES

(71) Applicant: GYRUS ACMI, INC. D/B/A OLYMPUS SURGICAL TECHNOLOGIES AMERICA, Westborough, MA (US)

(72) Inventors: William G. O’Neill, Maple Grove, MN (US); Kester Julian Batchelor, Mound, MN (US)

(21) Appl. No.: 18/837,670

(22) PCT Filed: Feb. 13, 2023
(86) PCT No.: PCT/US2023/062483

§ 371 Date: Aug. 12, 2024

Related U.S. Application Data
(60) Provisional application No. 63/268,086, filed on Feb. 16, 2022.

(51) Int. Cl. A61B 18/14

(52) U.S. Cl. CPC A61B 18/1482

(57) ABSTRACT

A surgical device to deliver energy to tissue can include a hand piece and a shaft. The hand piece can be operable to control the surgical device. The shaft can extend along a longitudinal axis and the shaft can include a proximal portion and a distal portion opposite the proximal portion. The proximal portion can be connected to the hand piece. The device can include an end effector connected to the distal portion of the shaft. The end effector can include an active electrode, an insulator at least partially surrounding the active electrode, and a return electrode adjacent the insulator. The device can include a heat pipe connected to the return electrode and extending at least partially through the shaft. The heat pipe can be configured to transfer heat from the return electrode to the hand piece.

### CLAIM OF PRIORITY

This patent application claims the benefit of priority to William G. O'Neill, U.S. Patent Application Ser. No. 63/268,086, entitled “HEAT TRANSFER TECHNIQUES IN HEAT-GENERATING MEDICAL DEVICES,” filed on Feb. 16, 2022, which is hereby incorporated by reference herein in its entirety.

### BACKGROUND

Tissue or organs of the human body often require surgical intervention which can include removal of tissue, organs, or portions thereof. In some cases, organs or tissue can be removed for sample collection, such as for a biopsy, in other examples, organs or tissue can be removed to address one or more problems or symptoms experienced by a patient. In either case, specialized surgical instruments can be used to safely and efficiently remove the organs or tissue.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an isometric view of a surgical system.

FIG. 2 illustrates a schematic view of a portion of a surgical system.

FIG. 3 illustrates an isometric view of a portion of a surgical device.

FIG. 4 illustrates an exploded view of a portion of a surgical device.

FIG. 5 illustrates an isometric view of a portion of a surgical device.

FIG. 6 illustrates an isometric view of a portion of a surgical device.

FIG. 7A illustrates an isometric view of a portion of a surgical device.

FIG. 7B illustrates an isometric view of a portion of a surgical device.

FIG. 7C illustrates an exploded isometric view of a portion of a surgical device.

FIG. 7D illustrates an isometric view of a portion of a surgical device.

FIG. 7E illustrates an isometric view of a portion of a surgical device.

### DETAILED DESCRIPTION

Electrosurgical devices can be used for cutting or resecting tissue for various surgical procedures. These devices can use electrodes to perform partial or completely hemostatic cuts or resections of the tissue using electrical energy (or radiofrequncy (RF) energy or ultra-high frequency (UHF) energy) delivered to the tissue through an active electrode. A passive electrode can receive the electrical energy from the tissue, allowing energy to flow through the tissue to perform the cut or resection. The electrodes can be separated by one or more dielectric components to help ensure energy flows through the tissue. These electrodes can heat up significantly during cutting or resection due to flowing energy and due to contact between the electrodes and relatively hot tissues. Excessive heating of the electrodes during use can cause overheating, which can lead to dielectric breakdown, dielectric melting, tissue necrosis, or other issues. Currently, devices such as the J-Hook™ cutting and coagulation device, use long electrode return arms to address these issues. The return electrode arms extend through the shaft and connect to a handpiece of the instrument. The return arms can serve as heat sinks to aid in cooling the cutting tip to help reduce overheating of the electrodes. Though effective, these return arms can be made of highly conductive materials, such as copper, and can be relatively expensive to manufacture and assemble.

The present disclosure helps to address these issues by including a heat pipe connected to one or more of the return electrodes and connected to the hand piece to transfer heat from the cutting tip to the hand piece. In one example, the device can help to reduce cost of the device by reducing the size of the return electrodes. In the same or another example, the device can also help to provide a hemostatic cut on both sides of the cut using a bipolar cutting tip that includes multiple return electrodes (on each side of the active electrode), where each of the return electrodes can be connected to the heat pipe to help transfer heat away from the cutting tip and to the hand piece. In another example, the device can include a single return electrode defining a medial slot for the active electrode, allowing for hemostatic cuts to be performed using a monopolar cutting tip.

The above discussion is intended to provide an overview of subject matter of the present patent application. It is not intended to provide an exclusive or exhaustive explanation of the invention. The description below is included to provide further information about the present patent application.

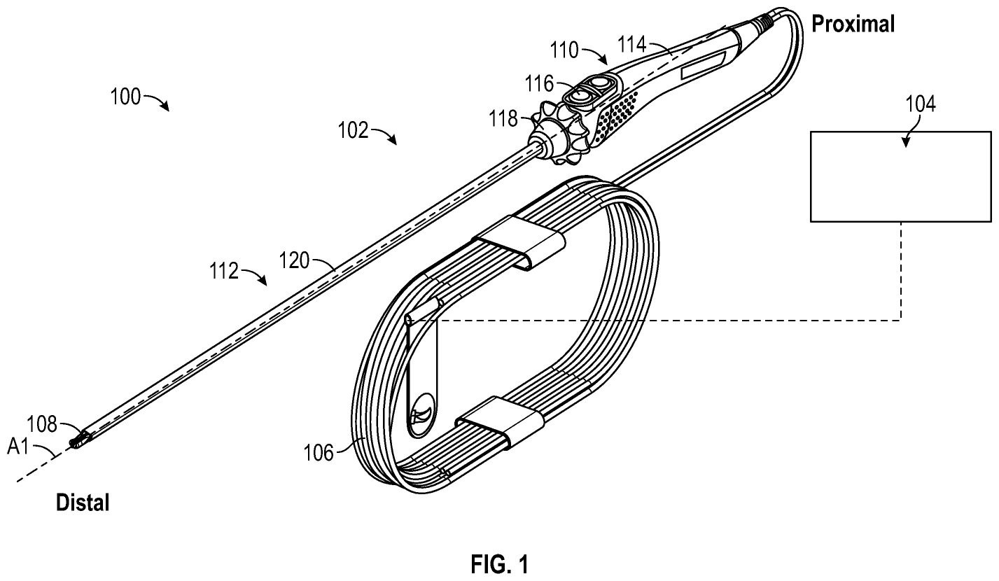

FIG. 1 illustrates an isometric view of a surgical system 100. The surgical system 100 can include a surgical instrument 102 that can be connected to a generator 104, such as via one or more wires or conductors 106. The generator 104 can be an electrical or electromagnetic device configured to deliver energy to the handpiece 110. The surgical instrument 102 can include an end effector 108, a handpiece 110, and an intermediate portion 112. The handpiece 110 can include a housing 114, controls 116, and a rotational actuator 118. FIG. 1 also shows orientation indicators Proximal and Distal and a longitudinal axis A1.

Generally, the handpiece 110 can be located at a proximal end of the instrument 102 and the end effector 108 can be located at a distal end of the surgical instrument 102. The intermediate portion 112 can extend between the handpiece 110 and the end effector 108 to operably couple the handpiece 110 to the end effector 108. Various movements of the end effector 108 can be controlled by one or more actuation systems of the handpiece 110. For example, the end effector 108 can be rotated along the longitudinal axis A1 of the surgical instrument 102. The handpiece 110 can also be used to operate the end effector 108 for cutting tissue, such as via delivery of electric or electromagnetic energy to tissue.

The housing 114 can be a frame that provides structural support between components of the surgical instrument 102. The housing 114 is shown as housing at least a portion of the actuation systems associated with the handpiece 110 for operating the end effector 108. However, some or all of the components need not be housed within the housing 114. The housing 114 can provide a rigid or semi-rigid structure for attachment of components, but the housing 114 does not necessarily house the components completely, or can house a portion of one or more of the components.

The intermediate portion 112 can include a shaft 120 that can extend between the housing 114 and the end effector 108 and can be configured to support one or more components therein. The controls 116 can be coupled to the housing 114 and can include or be connected to electronic circuitry within the housing 114, such as to connect the controls 116 to the generator 104. Such circuitry can send or transmit electric or electromagnetic energy to the end effector 108. In some examples, the electronic circuitry may reside outside the housing 114 but can be operably coupled to the housing 114 and the end effector 108.

In operation of some examples, a user can use the rotational actuator 118 to orient or rotate the end effector 108 as desired, such as to contact tissue of a patient. When it is desired to resect or cut the tissue, the user can operate the controls 116 to cause an electromagnetic energy, electric energy, or in some examples, ultrasonic energy, to be delivered to the end effector 108, such as to an electrode thereof, and to the tissue. Application of such energy can be used to cut and seal (e.g., hemostatically cut) or otherwise affect the tissue engaged with the end effector 108. In some examples, the energy can cause tissue to be coagulated, sealed, ablated, or can cause controlled necrosis. Throughout use of the 108 for such actions, the rotational actuator 118 can be operated to orient the end effector 108 as desired. Such a process can be repeated as desired or necessary to resect or treat an affected area.

Additional details of the end effector 108 and the intermediate portion 112 are discussed in the various examples below.

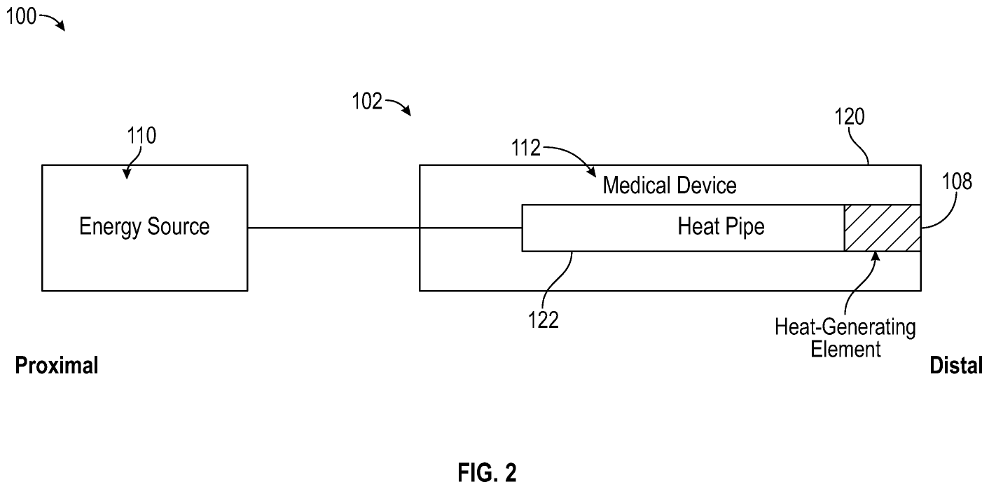

FIG. 2 illustrates a schematic view of a portion of the surgical system 100. The system 100 can be consistent with the system 100 discussed above; FIG. 2 shows additional details of the surgical system 100, such as in schematic form. FIG. 2 also shows orientation indicators Proximal and Distal.

More specifically, FIG. 2 shows the surgical system 100 including an energy source, such as the handpiece 110 (or the generator 104) connected to the intermediate portion 112, and shows the end effector 108 connected to a distal portion of the intermediate portion 112. FIG. 2 further shows a heat pipe 122 located at least partially within the shaft 120 and connected to the end effector 108. The heat pipe 122 can also be connected to the handpiece 110, such as to transfer heat between the end effector 108 and the handpiece 110 (or the housing 114), where heat transferred to the housing 114 can be rejected by the handpiece 110, the housing 114, or the generator 104.

The heat pipe 122 can generally be an elongate heat exchanger configured to passively transfer heat from the end effector 108 to the handpiece 110 or another component. The heat pipe 122 can be a passive heat pipe including one or more outer walls supporting one or more fluids therein. The heat pipe 122 can be optionally sealed, such as under vacuum or a pressure below atmospheric pressure. The fluid can be a refrigerant configured to changes phases, such as a refrigerant, or can be configured to exchange heat without changing phases. The fluid can include one or more of water, alcohol, refrigerant, or the like.

Optionally, the heat pipe 122 can include one or more chambers or a wick to promote heat transfer and fluid movement from a distal end of the heat pipe 122 to a proximal end of the heat pipe 122. Optionally, the heat pipe 122 can include one or more active components to further promote heat transfer and motivate fluid, such as a pump. The heat pipe 122 can also be other types of heat transfer devices, such as a as a heat exchanger, such as where a cooling fluid (e.g., water or glycol) is circulated through the heat pipe 122. Optionally, the heat pipe 122 can be an active cooling device, such as a thermo-electric cooler or Peltier device.

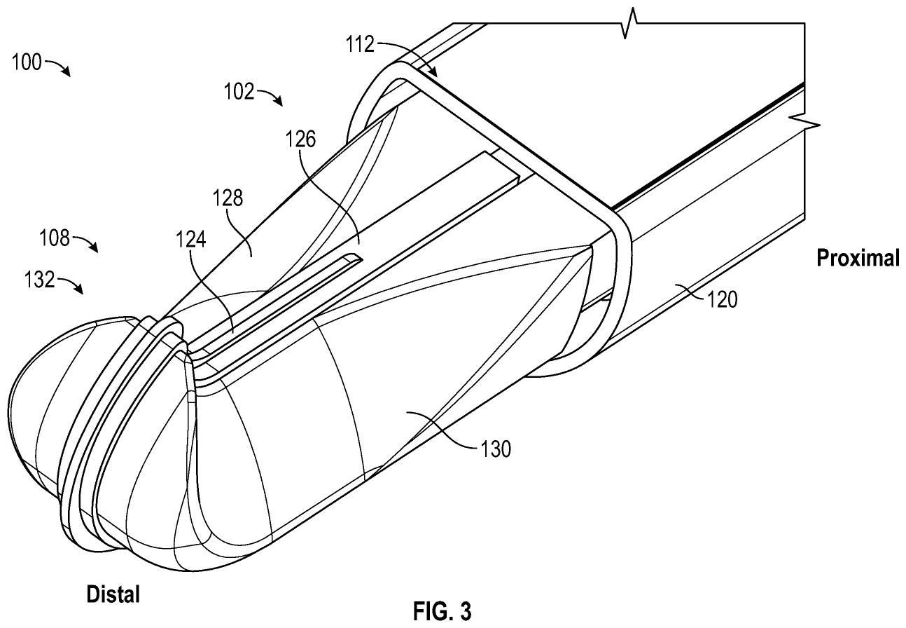

FIG. 3 illustrates an isometric view of the surgical instrument 102 of the surgical system 100 and shows orientation indicators Proximal and Distal. The surgical instrument 102 can be consistent with the surgical instrument 102 of FIGS. 1 and 2 discussed above; FIG. 3 shows additional details of the surgical instrument 102.

For example, FIG. 3 shows that the end effector 108 can be connected to a distal portion of the shaft 120 of the intermediate portion 112. FIG. 3 also shows that the end effector 108 can include an active electrode 124, an insulator 126, a return electrode 128, and a return electrode 130. Together, the active electrode 124, the insulator 126, the return electrode 128, and the return electrode 130 can form a cutting tip 132 that can be operable (e.g., using the handpiece 110) to cut or resect tissue.

The active electrode 124 can be a conductor made of one or more conductive materials, such as copper, silver, aluminum, platinum, gold, steel alloys, or the like. The active electrode 124 can be relatively thin to deliver a high current density from the generator 104 to tissue for efficient cutting and sealing of tissue. The return electrode 128 and 130 can be similarly constructed of one or more conductive materials. The active electrode 124 can be at least partially surrounded by the insulator 126, which can be a dielectric made of one or more insulative materials such as a ceramic, polymer, rubber, glass composites, or the like. The active electrode 124 can be or can include a heat-generating element, such as an RF sealing or other RF device, an electrosurgical cutting element, a resistive cutting element, a plasma-based device, laser device, ultrasonic device, or the like.

The insulator 126 can be sized and shaped to space the active electrode 124 away from the return electrons 128 and 130 such as to optimize cutting and sealing efficiency and effectiveness while helping to limit overheating of the cutting tip 132 and helping to limit arcing directly from the active electrode 124 to the return electrode 128 or the return electrode 130, helping to ensure electric power flows from the active electrode 124 to tissue before returning to the return electrode 128 or the return electrode 130. The active electrode 124, the insulator 126, the return electrode 128, and the return electrode 130 can be sized and shaped to form the cutting tip 132 that can be a hook or a J-hook cutting tip (or to have a hook shape or a J-hook shape), which can be a useful or efficient shape for cutting or resecting or sealing tissue electrosurgically.

The return electrode 128 and the return electrode 130 can be relatively large to help dissipate or reject heat and to help ensure there is adequate contact between the return electrode 128 and the return electrode 130 and tissue being cut or resected. Heat from the return electrode 128 can be transferred back to the handpiece 110 through the intermediate portion 112 (or the shaft 120) such as through the heat pipe 122, as discussed in further detail below.

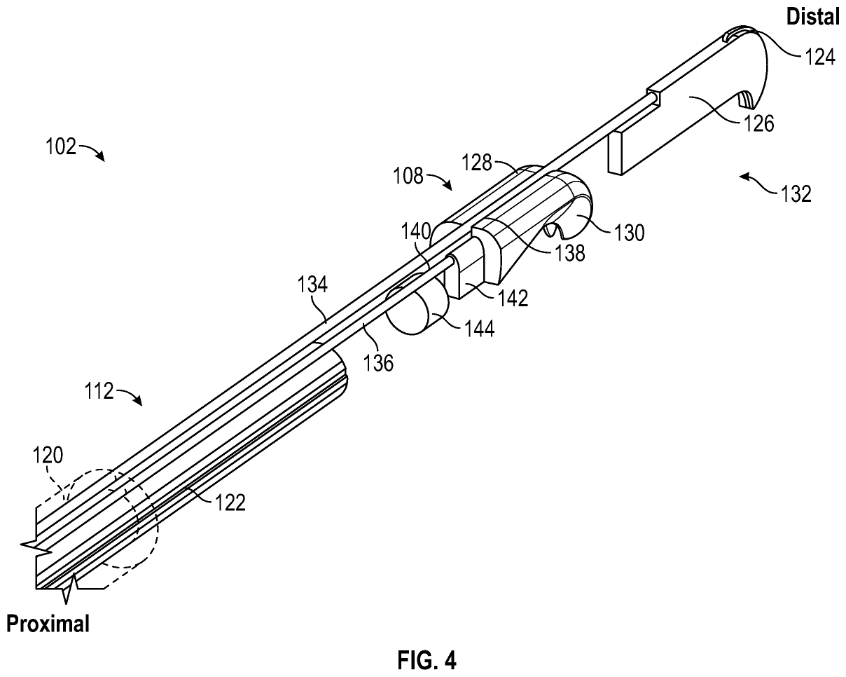

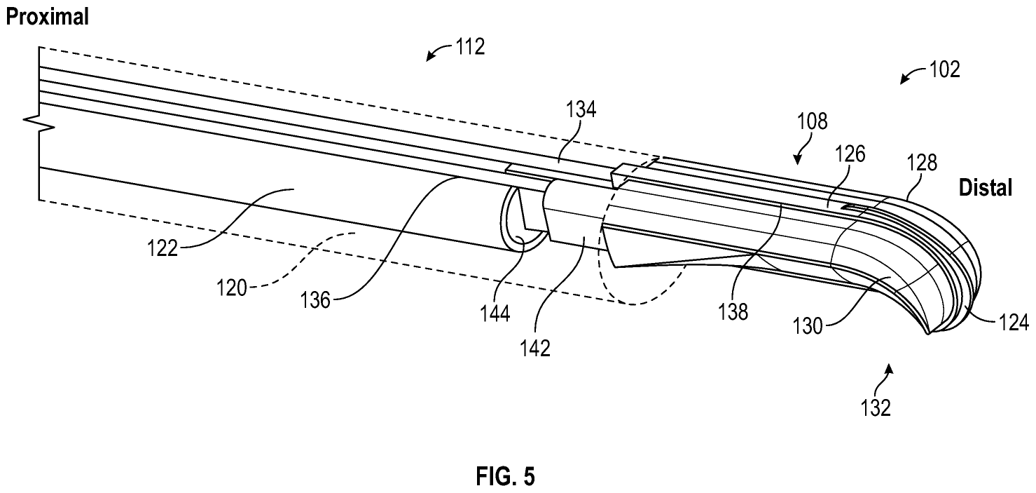

FIG. 4 illustrates an exploded view of a portion of the surgical device 102. FIG. 5 illustrates an isometric view of a portion of the surgical device 102. FIG. 6 illustrates an isometric view of a portion of the surgical device 102. FIGS. 4-6 are discussed together below. The surgical instrument 102 of FIGS. 4-6 can be consistent with the surgical instrument 102 discussed above. FIGS. 4-6 show additional details of the surgical instrument 102 and also show orientation indicators Proximal and Distal.

For example, FIGS. 4-6 show that the heat pipe 122 can be located at least partially within the shaft 120, which can be a hollow shaft or a tube. FIG. 4 also shows that the active electrode 124 can be connected to a conductor 134, such as an insulated wire. Similarly, the return electrode 128 or the return electrode 130 can be connected to a conductor 136, which can be an insulated wire or the like. The conductors 134 and 136 can extend through the shaft 120 to connect to the handpiece 110, such as to the controls 116 thereof. In such an arrangement, the active electrode 124 and the return electrodes 128 and 130 can form a monopolar cutting tip 132; however, other polarities can be used in other examples, as discussed in further detail below.

The return electrode 128 and the return electrode 130 can each be connected to each other and can be connected to a single conductor such that the return electrode 128 an the return electrode 130 together form a single return electrode, as shown in FIG. 4. In the example of FIG. 4, the return electrode 128 and the return electrode 130 can be connected to each other. The return electrode 128 and the return electrode 130 can be integrally formed or can be fastened adhered. In any of these examples, the return electrode 128 and the return electrode 130 can be split such as to form a slot 138 (or a gap) therebetween, such that the return electrode 128 and the return electrode 130 have a U-shape from a top perspective with respect to FIG. 4.

The slot 138 can be configured to receive and support the active electrode 124 and the insulator 126 at least partially therein such that the return electrode 128 engages or is adjacent a first side of the insulator 126 and the return electrode 130 engages or is adjacent a second side of the insulator 126. Optionally, the active electrode 124 and the insulator 126 can be fastened or adhered to the return electrode 128 and the return electrode 130 to form the cutting tip 132. The active electrode 124 and the insulator 126 can be positioned medially between the return electrode 128 and the return electrode 130, but can be positioned off-axis or asymmetrically in other examples.

The return electrode 128 can also include a base 140 (shown most clearly in FIG. 6) and the return electrode 130 can include a base 142 (shown most clearly in FIG. 5), where the base 140 and the base 142 can be located at a proximal portion of the return electrode 128 and the return electrode 130, respectively. Optionally, the base 140 or the base 142 can be connected to the conductor 136.

The end effector 108 or the surgical instrument 102 can also include a plug 144, which can be made of one or more conductive materials, such as copper, silver, aluminum, platinum, gold, steel alloys, or the like. The plug 144 can be inserted or located at least partially within a distal end of the heat pipe 122 such as to form a seal in the heat pipe 122, as shown in FIG. 5. FIG. 5 shows the plug 144 and base 142 held out of contact to more clearly show that the plug 144 can form a seal for the heat pipe

As shown in FIG. 6, the base 140 (and the base 142) can connect to or can contact the plug 144 such as to allow for heat to be transferred between the base 140, the base 142, and the plug 144, such as to transfer heat from the return electrode 128 and the return electrode 130 to the heat pipe 122. The plug 144 can have a cylindrical shape such as to allow for insertion of the plug 144 into the heat pipe 122. The plug 144 can have other shapes or cross-sectional shapes in other examples.

In operation of some examples, the handpiece 110 can be used to operate the cutting tip 132 to cut or resect tissue, which can generate heat in the return electrode 128 and the return electrode 130. For example, when an electrosurgical device has its tip or distal end heated via RF, resistive, UHG, electromagnetic, electric, or other techniques, heat from the return electrodes 128 and 130 can be transferred to the plug 144 and to the heat pipe 122, in order to reduce or maintain a safe and functional operating temperature of the cutting tip 132. Heat transferred to the heat pipe 122 can be transferred to fluid within the heat pipe 122, which can evaporate or can move away from the distal end of the heat pipe 122 to a proximal end of the heat pipe 12. At the proximal of the heat pipe 122, the fluid can transfer heat through a heat sink or other thermally conductive media or fluid within the handpiece 110 or the generator 104. As the fluid cools, the fluid can return to the distal end of the heat pipe 122, such as via a wicking structure, using capillary force, via grooves, or via other fluid conduit.

Use of the heat pipe 122 for cooling can be an efficient and cost-effective way to remove heat from the cutting tip 132. Effective heat reduction using the heat pipe 122 can also enable the surgical instrument 102 be used with relatively higher power or higher activation time of the active electrode 124, as the heat pipe 122 can transfer the heat proximally away from the tip more efficiently than standard heat sinks or other heat transfer elements.

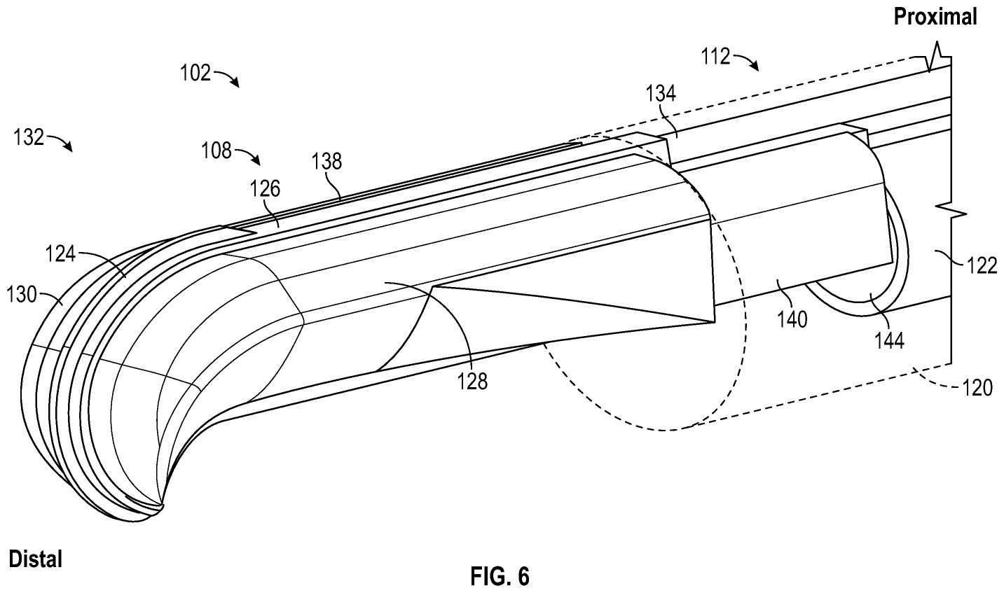

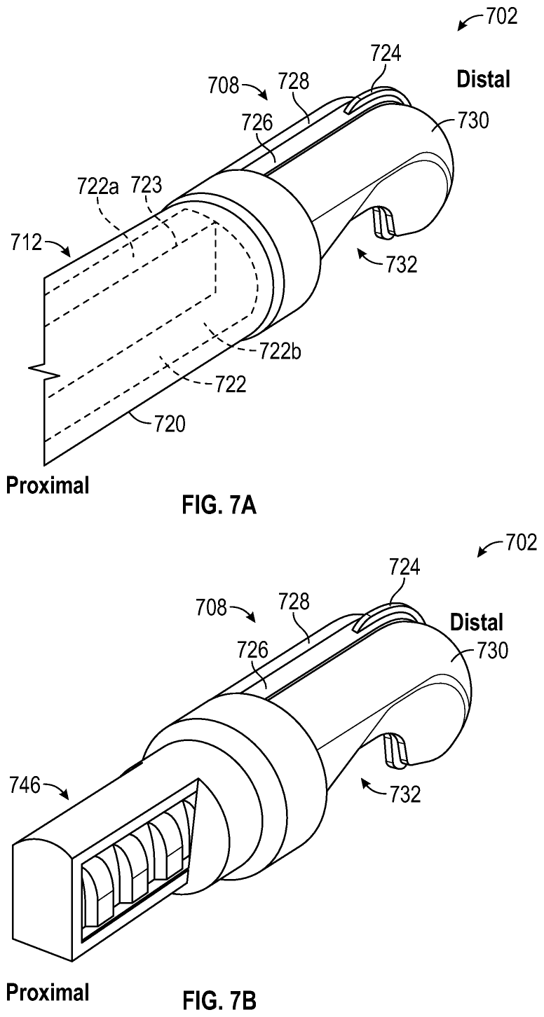

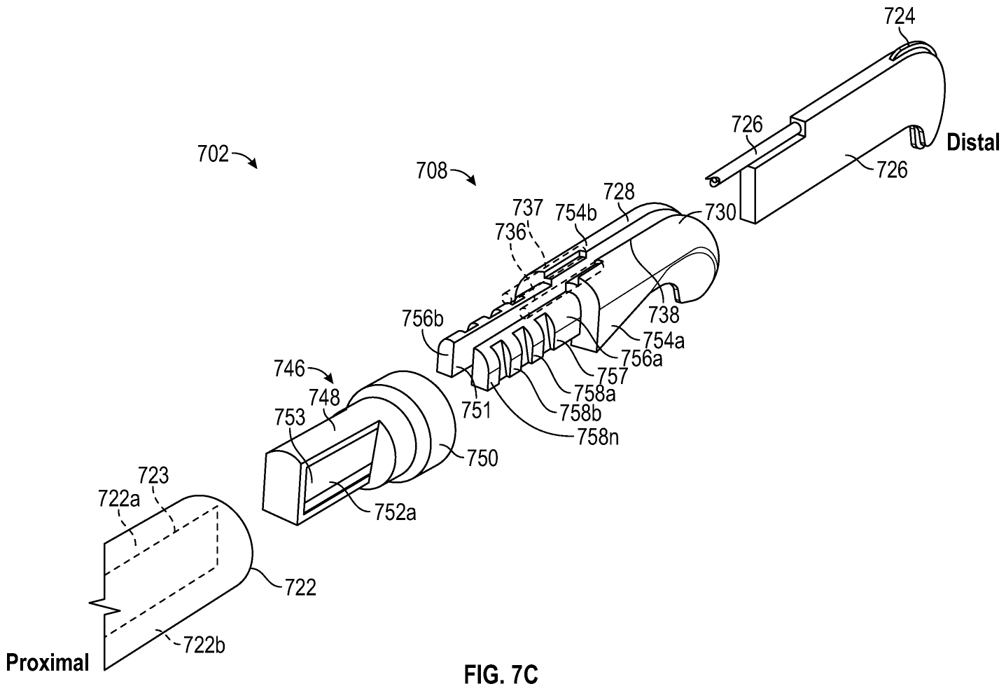

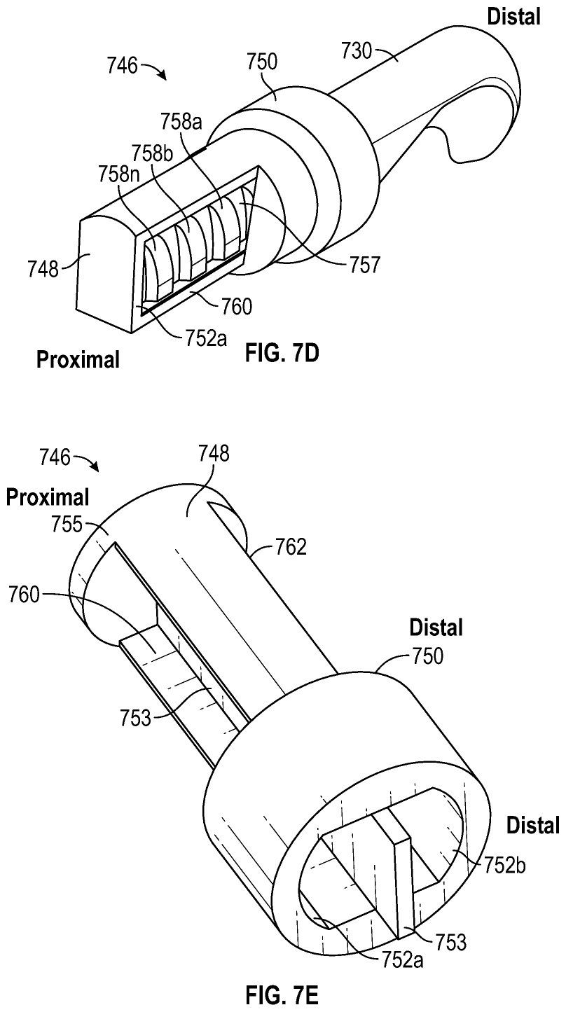

FIG. 7A illustrates an isometric view of a portion of a surgical device 702. FIG. 7B illustrates an isometric view of a portion of the surgical device 702. FIGS. 7A and 7B are discussed together below and also show orientation indicators Proximal and Distal. The surgical device 702 can be similar to the surgical device 102 discussed above; the surgical device 702 can include separate or discreet return electrodes and a plug assembly that allows for direct contact between a heat pipe and the return electrodes. Any of the surgical devices discussed above or below can be modified to include the features of the surgical device 702.

More specifically, the surgical device 702 can be similar to the surgical instrument 102 discussed above, in that the surgical device 702 can be connected to a generator (e.g., the generator 104), and can include a handpiece (e.g., the handpiece 110) connected to a shaft 720, which can support a heat pipe 722 at least partially therein. The heat pipe 722 can be similar to the heat pipe 122, but can include a divider 723, which can be an electrical insulator (such as made of one or more of polymer, glass composites, silicone rubber, ceramics, or the like). The divider 723 can form chambers 722a and 722b therein, which can act as independent (and electrically isolated) heat pipes such that the return electrodes 728 and 730 can remain electrically isolated through the heat pipe 722. An end effector 708 can be connected to a distal portion of the shaft 720 and can include an active electrode 724, an insulator 726, and return electrodes 728 and 730, which can at least partially define a cutting tip 732.

The surgical device 702 can also include a plug 746 that can be located or inserted at least partially within the shaft 720 and at least partially within the heat pipe 722. Optionally, the shaft 720 can be omitted and the heat pipe 722 can operate as the shaft. As discussed in further detail below, the return electrodes 728 and 730 can be independent components insertable into (or located at least partially within) the plug 746, such that at least a portion of the return electrodes 728 and 730 are located within the heat pipe 722. The plug 746 can be an insulator made of one or more of ceramic, polymer, glass composites, or the like.

FIG. 7C illustrates an exploded isometric view of a portion of the surgical device 702 and shows orientation indicators Proximal and Distal. The surgical device 702 of FIG. 7C can be consistent with the surgical device 702 of FIGS. 7A and 7B discussed above; FIG. 7C shows additional details of the surgical device 702.

For example, FIG. 7C shows that the return electrodes 728 and 730 can be separate components defining a gap 738 therebetween, which can be configured such that the active electrode 724 and the insulator 726 can be located at least partially within the gap 738 between the return electrodes 728 and 730. The active electrode 724 can be connected to a handpiece via a conductor 726. Each of the return electrodes 728 and 730 can be connected to the handpiece via conductors 736 and 737, respectively. The conductors 726, 736, and 737 can be wires or the like. In such an arrangement, the active electrode 724 and the return electrodes 728 and 730 can form a bipolar cutting tip 732.

FIG. 7C also shows the surgical device 702 with the shaft 720 removed and the components of the end effector 708 separated. FIG. 7C more clearly shows that the plug 746 can be a separate component configured to be inserted or located at least partially within the heat pipe 722. The plug 746 can include a body 748 and a collar 750 connected to a distal portion of the body 748. The body 748 can be located at least partially within (or can be at least partially insertable into) the heat pipe 722. The body 748 can receive a portion of the divider 723 therein or the body 748 can be insertable into the divider 723. The collar 750 can be engageable with a distal portion of the heat pipe 722 such as to form a seal between the heat pipe 722 and the plug 746. The body 748 and the collar 750 can together define slots 752a and 752b extending at least partially into the body 748 and configured to interface with the return electrodes 728 and 730, as discussed in further detail below.

FIG. 7C also shows that each of the conductors 728 and 730 can include engagement portions 754a and 754b, respectively, and proximal portions 756a and 756b, respectively. The proximal portion 756 of each conductor can be connected to (or integrally formed with) the engagement portion 754. The slots 752a and 752b can be separated by a divider 753. The divider 753 can extend into an opening 751, which can be formed between the proximal portions 756a and 756b.

The engagement portions 754 can be engageable with tissue. Each proximal portion 756 can include or can define a base 757 and one or more fins 758a-758n. Though three fins 758 are shown, each proximal portion 756 can include 1, 2, 4, 5, 6,7, 8, 9, 10, 15, or the like fins. The fins 758 can be configured to transfer heat from the return electrodes 728 and 730 to fluid within the heat pipe 722.

In this configuration, the end effector 708 can include multiple return electrodes, i.e., the electrodes 728 and 730, to provide a user with the ability to use the cutting tip 732 to perform hemostatic cuts or resections on both sides of the active electrode 724, helping to perform effective and efficient procedures. Additionally, through the plug 746, each of the return electrodes 728 and 730 can be connected to the heat pipe 722, which can allow the heat pipe 722 to reject heat from the return electrodes 728 and 730 (such as via the fins 758), helping to maintain the return electrodes 728 and 730 at a safe or effective operating temperature during cutting operations.

Optionally, the divider 723 can be omitted, and the heat pipe 722 can include an open chamber where the proximal portions 756a and 756b can be electrically isolated by one or more dielectrics within the heat pipe 722, such as air, and can the proximal portions 756a and 756b can be electrically isolated by distance or separation between the proximal portions 756a and 756b within the heat pipe 722. In one example where the divider 723 is omitted, the heat pipe 722 can use pure water (H2O) as a working heat transfer medium, as pure water (without minerals, etc.) can function as an electrical insulator.

The plug 746 can also maintain separation between the return electrodes 728 and 730 such that the return electrodes 728 and 730 do not contact each other within the plug 746. The plug 746 can also expose the return electrode 728 to only the chamber 722a and the return electrode 730 to only the chamber 722b. Because the plug 746 can be an insulator and because the chambers 722a and 722b can be isolated, the plug 746 and the heat pipe 722 can allow the cutting tip 732 to operate as a bipolar cutting tip. In another example, the shaft 720 can include two separate heat pipes therein.

FIG. 7D illustrates an isometric view of a portion of the surgical device 702. FIG. 7E illustrates an isometric view of a portion of the surgical device 702. FIGS. 7D and 7E are discussed together below and also show orientation indicators Proximal and Distal. The surgical device 702 of FIGS. 7D and 7E can be consistent with the surgical device 702 of FIGS. 7A-7C discussed above. FIGS. 7D and 7E show additional details of the surgical device 702.

For example, FIG. 7D shows that the return electrode 728 can be removed from the 746, such that the return electrodes 728 and 730 can be independent components. FIG. 7E shows the plug 746 with both of the return electrodes 728 and 730, revealing the slots or channels 752a and 752b that can extend through the collar 750 and into the body 748. The slots 752 can be separated by the divider 753, which can extend from a proximal cap 755 (of the body 748) to a distal end or portion or termination of the plug 746.

FIGS. 7D and 7E also show that the body 748 can define or include windows 760 and 762, which can extend at least partially through the body 748. The windows 760 and 762 can be configured to expose at least a portion of the return electrodes 728 and 730 when the return electrodes 728 and 730 are inserted into the plug 746. For example, as shown in FIG. 7D, when the return electrode 728 is inserted into the slot 752a, the base 757 and the fins 758 can extend at least partially through the windows 760 or can be at least partially exposed thereby. In this way, the base 757 and the fins 758 can be in direct contact with fluid within the heat pipe 722 when the plug 746 is inserted into or located in the heat pipe 722, helping to improve heat transfer efficiency or effectiveness between the return electrodes 728 and 730 and the heat pipe 722.

In this way, the surgical device 702 can use a bipolar cutting tip 732 that is actively cooled via the heat pipe 722 to perform hemostatic cuts or resections on tissue, where the bipolar arrangement can allow for deeper or cleaner cuts and the heat pipe 722 can allow for relatively low-cost heat transfer from the return electrodes 728 and 730 to allow the cutting tip 732 to perform efficiently or effectively.

The techniques of this disclosure may be used in medical devices that include heat generation, such as a heat-generating element. Heat-generating elements may be associated with RF sealing or other RF devices, electrosurgical cutting elements, resistive cutting elements, plasma-based devices, laser devices, ultrasonic devices, etc. Another example of a medical device having a heat-generating active element is the Thunderbeat™ hybrid energy medical instrument that utilizes ultrasonic energy, bipolar energy, or a combination of ultrasonic and bipolar energy, to dissect and seal tissue. The heat pipe (e.g., 122 or 722) can be used to transfer heat away from the tissue-contacting therapeutic element to reduce the chance of such unintentional tissue necrosis. Thus, the techniques described in this disclosure may be used in any type of heat-generating medical instrument (e.g., harmonic scalpel, monopolar or bipolar cutting, etc.).

### NOTES AND EXAMPLES

The following, non-limiting examples, detail certain aspects of the present subject matter to solve the challenges and provide the benefits discussed herein, among others.

Example 1 is a surgical device to deliver energy to tissue, the surgical device comprising: a hand piece operable to control the surgical device; a shaft extending along a longitudinal axis, the shaft including a proximal portion and a distal portion opposite the proximal portion, the proximal portion connected to the hand piece; an end effector connected to the distal portion of the shaft, the end effector comprising: an active electrode configured to receive energy from the hand piece and deliver the energy to tissue; an insulator at least partially surrounding the active electrode; and a return electrode adjacent the insulator, the return electrode configured to receive energy from the active electrode through tissue; and a heat pipe connected to the return electrode and extending at least partially through the shaft, the heat pipe configured to transfer heat from the return electrode to the hand piece.

In Example 2, the subject matter of Example 1 optionally includes wherein the return electrode is split to include a first portion and a second portion defining a gap therebetween, the insulator and the active electrode located at least partially within the gap between the first portion and the second portion.

In Example 3, the subject matter of Example 2 optionally includes wherein the active electrode and the insulator are positioned medially with respect to the first portion and the second portion of the return electrode.

In Example 4, the subject matter of any one or more of Examples 2-3 optionally include wherein the return electrode has a substantially U-shape.

In Example 5, the subject matter of any one or more of Examples 1-4 optionally include a plug connected to a proximal portion of the return electrode, the plug located at least partially within the heat pipe to transfer heat from the return electrode to the hand piece.

In Example 6, the subject matter of Example 5 optionally includes wherein the plug forms a seal of a distal portion of the heat pipe.

In Example 7, the subject matter of any one or more of Examples 5-6 optionally include wherein the plug is integrally formed with the return electrode.

In Example 8, the subject matter of any one or more of Examples 1-7 optionally include wherein the return electrode includes a first return electrode connected to a first side of the insulator, and wherein the surgical device includes a second return electrode connected to a second side of the insulator.

In Example 9, the subject matter of Example 8 optionally includes wherein the first return electrode is connected to the heat pipe, and wherein the second return electrode is connected to the heat pipe.

In Example 10, the subject matter of any one or more of Examples 8-9 optionally include a plug connected to a proximal portion of the first return electrode and a proximal portion of the second return electrode, the plug located at least partially within the heat pipe to transfer heat from the first return electrode and the second return electrode to the hand piece.

In Example 11, the subject matter of Example 10 optionally includes wherein the proximal portion of the first return electrode extends at least partially into a first channel of the plug, and wherein the proximal portion of the second return electrode extends at least partially into a second channel of the plug.

In Example 12, the subject matter of Example 11 optionally includes wherein the proximal portion of the first return electrode includes a base and includes a plurality of fins extending laterally from the base to transfer heat from the first return electrode to the heat pipe.

In Example 13, the subject matter of any one or more of Examples 1-12 optionally include wherein the active electrode and the return electrode together define a cutting tip having a hook-shape.

Example 14 is a surgical device to deliver energy to tissue, the surgical device comprising: a hand piece operable to control the surgical device; a shaft extending along a longitudinal axis, the shaft including a proximal portion and a distal portion opposite the proximal portion, the proximal portion connected to the hand piece; an end effector connected to the distal portion of the shaft, the end effector comprising: a return electrode including a first portion and a second portion defining a slot therebetween; an active electrode located at least partially within the slot, the active electrode configured to receive energy from the hand piece and deliver the energy to tissue; and an insulator located at least partially within the slot and at least partially surrounding the active electrode; and a heat pipe connected to the first portion and the second portion of the return electrode, the heat pipe extending at least partially through the shaft, and the heat pipe configured to transfer heat from the return electrode to the hand piece.

In Example 15, the subject matter of Example 14 optionally includes wherein the active electrode and the insulator are positioned medially with respect to the first portion and the second portion of the return electrode.

In Example 16, the subject matter of any one or more of Examples 14-15 optionally include a plug connected to the first portion and the second portion of the return electrode, the plug located at least partially within the heat pipe to transfer heat from the return electrode to the hand piece.

In Example 17, the subject matter of Example 16 optionally includes wherein the plug is integrally formed with the return electrode.

Example 18 is a surgical device to deliver energy to tissue, the surgical device comprising: a hand piece operable to control the surgical device; an end effector connected to the hand piece, the end effector comprising: a first return electrode and a second return electrode forming a gap therebetween; and an active electrode located at least partially within the gap, the active electrode configured to receive energy from the hand piece and deliver the energy to tissue; and a heat pipe connected to the first return electrode and the second return electrode, the heat pipe connected to the hand piece to transfer heat from at least one of the first return electrode and the second return electrode to the hand piece.

In Example 19, the subject matter of Example 18 optionally includes a plug connected to a proximal portion of the first return electrode and a proximal portion of the second return electrode, the plug located at least partially within the heat pipe to transfer heat from the first return electrode and the second return electrode to the hand piece.

In Example 20, the subject matter of Example 19 optionally includes wherein the proximal portion of the first return electrode extends at least partially into a first channel of the plug, and wherein the proximal portion of the second return electrode extends at least partially into a second channel of the plug.

In Example 21, the subject matter of Example 20 optionally includes wherein the proximal portion of the first return electrode includes a base and includes a plurality of fins extending laterally from the base to transfer heat from the first return electrode to the heat pipe.

Example 22 is a surgical device to deliver energy to tissue, the surgical device comprising: a hand piece operable to control the surgical device; a shaft extending along a longitudinal axis, the shaft including a proximal portion and a distal portion opposite the proximal portion, the proximal portion connected to the hand piece; an end effector connected to the distal portion of the shaft, the end effector comprising: a return electrode including a first portion and a second portion defining a slot therebetween; an active electrode located at least partially within the slot, the active electrode configured to receive energy from the hand piece and deliver the energy to tissue; and an insulator located at least partially within the slot and at least partially surrounding the active electrode.

In Example 23, the subject matter of Example 22 optionally includes wherein the active electrode and the insulator are positioned medially with respect to the first portion and the second portion of the return electrode.

In Example 24, the subject matter of any one or more of Examples 22-23 optionally include a heat pipe connected to the first portion and the second portion of the return electrode, the heat pipe extending at least partially through the shaft, and the heat pipe configured to transfer heat from the return electrode to the hand piece.

In Example 25, the subject matter of Example 24 optionally includes a plug connected to the first portion and the second portion of the return electrode, the plug located at least partially within the heat pipe to transfer heat from the return electrode to the hand piece.

In Example 26, the subject matter of Example 25 optionally includes wherein the plug is integrally formed with the return electrode.

In Example 27, the apparatuses or method of any one or any combination of Examples 1-26 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical device to deliver energy to tissue, the surgical device comprising:
a hand piece operable to control the surgical device;
a shaft extending along a longitudinal axis, the shaft including a proximal portion and a distal portion opposite the proximal portion, the proximal portion connected to the hand piece;
an end effector connected to the distal portion of the shaft, the end effector comprising:
an active electrode configured to receive energy from the hand piece and deliver the energy to tissue;
an insulator at least partially surrounding the active electrode; and
a return electrode adjacent the insulator, the return electrode configured to receive energy from the active electrode through tissue; and
a heat pipe connected to the return electrode and extending at least partially through the shaft, the heat pipe configured to transfer heat from the return electrode to the hand piece.

2. The surgical device of claim 1, wherein the return electrode is split to include a first portion and a second portion defining a gap therebetween, the insulator and the active electrode located at least partially within the gap between the first portion and the second portion.

3. The surgical device of claim 2, wherein the active electrode and the insulator are positioned medially with respect to the first portion and the second portion of the return electrode.

4. The surgical device of claim 2, wherein the return electrode has a substantially U-shape.

5. The surgical device of claim 1, comprising:
a plug connected to a proximal portion of the return electrode, the plug located at least partially within the heat pipe to transfer heat from the return electrode to the hand piece.

6. The surgical device of claim 5, wherein the plug forms a seal of a distal portion of the heat pipe.

7. The surgical device of claim 5, wherein the plug is integrally formed with the return electrode.

8. The surgical device of claim 1, wherein the return electrode includes a first return electrode connected to a first side of the insulator, and wherein the surgical device includes a second return electrode connected to a second side of the insulator.

9. The surgical device of claim 8, wherein the first return electrode is connected to the heat pipe, and wherein the second return electrode is connected to the heat pipe.

10. The surgical device of claim 8, comprising:
a plug connected to a proximal portion of the first return electrode and a proximal portion of the second return electrode, the plug located at least partially within the heat pipe to transfer heat from the first return electrode and the second return electrode to the hand piece.

11. The surgical device of claim 10, wherein the proximal portion of the first return electrode extends at least partially into a first channel of the plug, and wherein the proximal portion of the second return electrode extends at least partially into a second channel of the plug.

12. The surgical device of claim 11, wherein the proximal portion of the first return electrode includes a base and includes a plurality of fins extending laterally from the base to transfer heat from the first return electrode to the heat pipe.

13. The surgical device of claim 1, wherein the active electrode and the return electrode together define a cutting tip having a hook-shape.

14. A surgical device to deliver energy to tissue, the surgical device comprising:
a hand piece operable to control the surgical device;
a shaft extending along a longitudinal axis, the shaft including a proximal portion and a distal portion opposite the proximal portion, the proximal portion connected to the hand piece;
an end effector connected to the distal portion of the shaft, the end effector comprising:
a return electrode including a first portion and a second portion defining a slot therebetween;
an active electrode located at least partially within the slot, the active electrode configured to receive energy from the hand piece and deliver the energy to tissue; and
an insulator located at least partially within the slot and at least partially surrounding the active electrode; and
a heat pipe connected to the first portion and the second portion of the return electrode, the heat pipe extending at least partially through the shaft, and the heat pipe configured to transfer heat from the return electrode to the hand piece.

15. The surgical device of claim 14, wherein the active electrode and the insulator are positioned medially with respect to the first portion and the second portion of the return electrode.

16. The surgical device of claim 14, comprising:
a plug connected to the first portion and the second portion of the return electrode, the plug located at least partially within the heat pipe to transfer heat from the return electrode to the hand piece.

17. The surgical device of claim 16, wherein the plug is integrally formed with the return electrode.

18. A surgical device to deliver energy to tissue, the surgical device comprising:
a hand piece operable to control the surgical device;
an end effector connected to the hand piece, the end effector comprising:
a first return electrode and a second return electrode forming a gap therebetween; and
an active electrode located at least partially within the gap, the active electrode configured to receive energy from the hand piece and deliver the energy to tissue; and
a heat pipe connected to the first return electrode and the second return electrode, the heat pipe connected to the hand piece to transfer heat from at least one of the first return electrode and the second return electrode to the hand piece.

19. The surgical device of claim 18, comprising:
a plug connected to a proximal portion of the first return electrode and a proximal portion of the second return electrode, the plug located at least partially within the heat pipe to transfer heat from the first return electrode and the second return electrode to the hand piece.

20. The surgical device of claim 19, wherein the proximal portion of the first return electrode extends at least partially into a first channel of the plug, and wherein the proximal portion of the second return electrode extends at least partially into a second channel of the plug.

21. The surgical device of claim 20, wherein the proximal portion of the first return electrode includes a base and includes a plurality of fins extending laterally from the base to transfer heat from the first return electrode to the heat pipe.
